(19) United States
(12) Patent Application Publication
(10) Pub. No.: US 2003/0113318 A1
(43) Pub. Date: Jun. 19, 2003

(54) TNF inhibition for the treatment of pre-menstrual syndrome and primary dysmenorrhea

(76) Inventor: Edward Lewis Tobinick, Los Angeles, CA (US)

Correspondence Address: EZRA SUTTON, P.A., PLAZA 9, 900 ROUTE 9, WOODBRIDGE, NJ, 07095, US

(21) Appl. No.: 10/340,890

(22) Filed: Jan. 13, 2003

Related U.S. Application Data
(63) Continuation-in-part of application No. 10/269,745, filed on Oct. 9, 2002.
Application No. 10/269,745 is a continuation-in-part of application No. 10/236,097, filed on Sep. 6, 2002.
Application No. 10/236,097 is a continuation-in-part of application No. 09/841,844, filed on Apr. 25, 2001, now Pat. No. 6,537,549.
Application No. 09/841,844 is a continuation-in-part of application No. 09/826,976, filed on Apr. 5, 2001, now Pat. No. 6,419,944.
Application No. 09/826,976 is a continuation-in-part of application No. 09/563,651, filed on May 2, 2000, now Pat. No. 6,471,961.
Application No. 09/563,651 is a continuation-in-part of application No. 09/476,643, filed on Dec. 31, 1999, now Pat. No. 6,177,077.
Application No. 09/476,643 is a continuation-in-part of application No. 09/275,070, filed on Mar. 23, 1999, now Pat. No. 6,015,557.
Application No. 09/275,070 is a continuation-in-part of application No. 09/256,388, filed on Feb. 24, 1999, now abandoned.

(51) Int. Cl. A61K 39/395

(52) U.S. Cl. 424/134.1; 424/145.1

(57) ABSTRACT

Methods for treating pre-menstrual syndrome and primary dysmenorrhea in humans by administering to the human a therapeutically effective dose of specific biologics are presented. The biologics of consideration include antagonists of tumor necrosis factor alpha. The administration of these biologics is performed by specific methods, including parenteral, intranasal, or anatomically localized administration designed for perispinal use. Anatomically localized administration involving perispinal use includes, but is not limited to the subcutaneous, intramuscular, interspinous, epidural, peridural, parenteral or intrathecal routes.

### RELATED APPLICATIONS

This is a continuation-in-part of application Ser. No. 10/269,745 filed on Oct. 9, 2002 which is a continuation-in-part of application Ser. No. 10/236,097, filed on Sep. 6, 2002, which is a continuation-in-part of application Ser. No. 09/841,844, filed on Apr. 25, 2001, which is a continuation-in-part of application Ser. No. 09/826,976, filed on Apr. 5, 2001, now U.S. Pat. No. 6,419,944, which is a continuation-in-part of application Ser. No. 09/563,651, filed on May 2, 2000, which is a continuation-in-part of application Ser. No. 09/476,643, filed on Dec. 31, 1999, now U.S. Pat. No. 6,177,077, which is a continuation-in-part of application Ser. No. 09/275,070, filed on Mar. 23, 1999, now U.S. Pat. No. 6,015,557, which is a continuation-in-part of application Ser. No. 09/256,388, filed on Feb. 24, 1999, now abandoned.

### FIELD OF THE INVENTION

The present invention relates to novel methods of use of specific cytokine antagonists for the treatment of pre-menstrual syndrome and primary dysmenorrhea. More particularly, these cytokine antagonists are used in a new treatment of pre-menstrual syndrome, primary dysmenorrhea, and related disorders (including related mood disorders) utilizing specific anatomic methods of administration of these specific biologics. The delivery of these cytokine antagonists is performed by specific methods, including parenteral administration, intranasal administration, or perispinal administration. Perispinal administration involves an anatomically localized injection performed so as to deliver the therapeutic molecule directly into the vicinity of the spine. Perispinal administration includes, but is not limited to the subcutaneous, intramuscular, interspinous, epidural, peridural, parenteral, or intrathecal routes, and may be remote from the ultimate site of pathology. Intranasal administration includes the delivery of these particular cytokine antagonists by instillation into the nasal passages, either by nasal spray or nasal inhaler. The cytokine antagonists of consideration are those designed to block the action of, inhibit, or antagonize the biologic effects of tumor necrosis factor-alpha (TNF). These antagonists may take the form of a fusion protein (such as etanercept); a monoclonal antibody (such as infliximab); a binding protein (such as onercept, Serono); an antibody fragment (such as CDP 870, Pharmacia); or other types of molecules which are potent, selective, and specific inhibitors of the action of these pro-inflammatory cytokines and are capable of being used by parenteral injection or by intranasal administration.

### BACKGROUND OF THE INVENTION

Localized administration for the treatment of localized clinical disorders has many clinical advantages over the use of conventional systemic treatment. Locally administered medication after delivery diffuses through local capillary, venous, arterial, and lymphatic action to reach the anatomic site of pathology, or, alternatively, to reach the cerebrospinal fluid (CSF). In addition local administration of a biologic in the vicinity of the spine (perispinal administration) has the key advantage of improved delivery of the agent to the central nervous system (CNS). Local intranasal administration of a biologic is another method to improve delivery of the biologic to the CNS, and is discussed here as a method to treat pre-menstrual syndrome, primary dysmenorrhea, and related disorders, utilizing TNF antagonists.

All of the cytokine antagonists which are currently available have been developed for systemic administration. This is because all were developed to treat systemic illnesses, including rheumatoid arthritis, juvenile rheumatoid arthritis, psoriatic arthritis, or Crohn's Disease.

The use of cytokine antagonists to treat neurological and neuropsychiatric disorders is discussed in several previous patents of this inventor, including U.S. Pat. Nos. 6,015,557, 6,177,077, 6,419,944 B2 and other pending applications of this inventor. This invention includes further applications of these ideas.

Perispinal administration of biologics when compared to systemic administration, carries with it one or more of the following advantages:

1) greater efficacy due to the achievement of higher local concentration;

2) greater efficacy due to the ability of the administered therapeutic molecule to reach the target tissue without degradation caused by hepatic or systemic circulation;

3) more rapid onset of action;

4) longer duration of action;

5) Potentially fewer side effects, due to lower required dosage;

6) greatly improved efficacy due to improved delivery of the therapeutic molecule to the CNS.

Clinical experience utilizing perispinal administration of etanercept for treating lumbar and cervical radiculopathy and other forms of neuropathic pain caused by vertebral disc disease has demonstrated the dramatic efficacy, and the extraordinarily rapid onset of action produced by perispinal administration of etanercept for these disorders. Perispinal administration of the other cytokine antagonists of consideration here, for treating other neurological disorders or for treating neuropsychiatric disorders, as partially enumerated above, shares the above advantages.

The therapeutic molecules of consideration here have many biologic effects. Etanercept, for example, in addition to being a potent anti-inflammatory also has important anti-apoptotic effects which may be of particular importance in treating neurodegenerative diseases, such as Alzheimer's Disease and Parkinson's Disease, where apoptosis plays a pathogenetic role.

Biologics have been developed which have been shown to offer dramatic clinical benefit for systemic illnesses in humans, even for those disorders which have not responded to large and repeated doses of corticosteroids. These biologics fall into the category of cytokine antagonists because they block, or antagonize, the biologic action of a specific cytokine which has adverse clinical effects. These cytokines include the pro-inflammatory cytokines interleukin-1 and TNF. For the purposes of this discussion, “antagonist”, “inhibitor”, and “blocker” are used interchangeably.

Specific inhibitors of TNF, only recently commercially available, now provide for therapeutic intervention in TNF mediated disorders. These agents have been developed to treat systemic illnesses, and therefore have been developed for systemic administration. Various biopharmaceutical companies have developed TNF antagonists to treat systemic illnesses: Immunex Corporation developed etanercept (Enbrel) to treat rheumatoid arthritis; Johnson and Johnson developed infliximab (Remicade) to treat Crohn's Disease and rheumatoid arthritis; D2E7, now known as adalimumab (Humira), a human anti-TNF monoclonal antibody (Abbott) is being developed to treat rheumatoid arthritis and Crohn's Disease; Celltech is developing CDP 571 to treat Crohn's Disease and CDP 870 to treat rheumatoid arthritis; and Serono is developing onercept, a recombinant TNF binding protein (r-TBP-1) for treating rheumatoid arthritis and psoriasis/psoriatic arthritis.

Recent research has demonstrated that a new TNF antagonist can be manufactured from an existing molecule by subtracting a portion of the amino acid sequence from the molecule. This has the advantage of making the molecule smaller. This smaller molecule can be easier to manufacture and may have clinical advantages, such as reduced immunogenicity in the human in vivo. Therefore, the molecules of consideration here shall also include, in addition to those specified, any molecule which contains a fragment of any of the named molecules. A fragment shall be defined as an identical amino acid sequence 50% or greater in length of the original molecule and possessing TNF binding capability.

### DESCRIPTION OF THE PRIOR ART

Pharmacologic chemical substances, compounds and agents which are used for the treatment of neurological disorders, trauma, injuries and compression having various organic structures and metabolic functions have been disclosed in the prior art. For example, U.S. Pat. Nos. 5,756,482 and 5,574,022 to ROBERTS et al disclose methods of attenuating physical damage to the nervous system and to the spinal cord after injury using steroid hormones or steroid precursors such as pregnenolone, and pregnenolone sulfate in conjunction with a non-steroidal anti-inflammatory substance such as indomethacin. These prior art patents do not teach the use of specific cytokine antagonists administered by the perispinal route as a way of treating neurological or neuropsychiatric disorders or diseases., as in the present invention.

U.S. Pat. No. 5,863,769 discloses using IL-1 RA for treating various diseases. However, it does not disclose administering cytokine antagonists by the perispinal route as a way of treating neurological or neuropsychiatric disorders or diseases.

U.S. Pat. No. 6,013,253 discloses using interferon and IL-1 RA for treating multiple sclerosis. However, it does not disclose administering cytokine antagonists by the perispinal route as a way of treating neurological or neuropsychiatric disorders or diseases.

U.S. Pat. No. 5,075,222 discloses the use of IL-1 inhibitors for treatment of various disorders. However, it does not disclose administering cytokine antagonists by the perispinal route as a way of treating neurological or neuropsychiatric disorders or diseases.

U.S. Pat. No. 6,159,460 discloses the use of IL-1 inhibitors for treatment of various disorders. However, it does not disclose administering cytokine antagonists by the perispinal route as a way of treating neurological or neuropsychiatric disorders or diseases.

U.S. Pat. No. 6,096,728 discloses the use of IL-1 inhibitors for treatment of various disorders. However, it does not disclose administering cytokine antagonists by the perispinal route as a way of treating neurological or neuropsychiatric disorders or diseases.

U.S. Pat. No. 6,277,969 discloses the use of anti-TNF antibodies for treatment of various disorders. However, it does not disclose administering cytokine antagonists by the perispinal route as a way of treating neurological or neuropsychiatric disorders or diseases.

U.S. Pat. No. 5,605,690 discloses the use of TNF inhibitors for treatment of various disorders. However, it does not disclose administering cytokine antagonists by the perispinal route as a way of treating neurological or neuropsychiatric disorders or diseases.

None of the prior art patents disclose or teach the use of localized administration of a cytokine antagonist as in the present invention as a way of treating neurological or neuropsychiatric disorders or diseases, in which the cytokine antagonist provides the patient with a better opportunity to heal, slows disease progression, or otherwise improves the patient's health.

Accordingly, it is an object of the present invention to provide a biologic administered through perispinal administration as a new method of pharmacologic treatment of premenstrual syndrome or primary dysmenorrhea such that the use of these biologics will result in the amelioration of these conditions.

Another object of the present invention is to provide cytokine antagonists for providing suppression and inhibition of the action of specific cytokines in a human to treat premenstrual syndrome or primary dysmenorrhea.

Another object of the present invention is to provide cytokine antagonists that produce biologic effects in patients with premenstrual syndrome or primary dysmenorrhea by inhibiting the action of specific cytokines in the human body for the immediate, short term (acute conditions) and long term (chronic conditions), such that these biologic effects will produce clinical improvement in the patient and will give the patient a better opportunity to heal, improve cognitive function, slow disease progression, reduce pain, or otherwise improve the patient's health.

Another object of the present invention is to provide cytokine antagonists, using anatomically localized administration in the vicinity of the spine as the preferred forms of administration, that provide therapeutic benefit utilizing either acute or chronic treatment regimens for treating premenstrual syndrome or primary dysmenorrhea.

### SUMMARY OF THE INVENTION

The present invention provides methods for treating pre-menstrual syndrome and primary dysmenorrhea in humans by administering to the human a therapeutically effective dose of a specific biologic. The biologics of consideration include antagonists of tumor necrosis factor-alpha. The administration of these biologics is performed by specific methods, including the categories of parenteral administration or anatomically localized administration involving perispinal or intranasal delivery. Anatomically localized administration involving perispinal use includes, but is not limited to the subcutaneous, intramuscular, interspinous, epidural, peridural, parenteral or intrathecal routes.

### DETAILED DESCRIPTION OF THE PREFERRED EMBODIMENTS

Perispinal administration is a novel new concept for a delivery method for cytokine antagonists for treating premenstrual syndrome or primary dysmenorrhea.

For the purposes of this discussion, “perispinal” means in the anatomic vicinity of the spine. For this discussion “anatomic vicinity” is generally defined as within 10 centimeters, or functionally defined as in close enough anatomic proximity to allow the therapeutic molecules of consideration herein to reach the spine and/or the subarachnoid space surrounding the spinal cord in therapeutic concentration when administered directly to this area. For the treatment of brain disorders perispinal administration is effective because it delivers the biologic to the CNS in a therapeutic amount. This is accomplished through enhanced delivery of the therapeutic molecule to the CNS, either by direct diffusion or via enhanced delivery into the cerebrospinal fluid (CSF) which is present in the thecal sac. This usually occurs without direct intrathecal injection, but rather by diffusion from the peridural space into the subarachnoid space. Direct injection of these specific cytokine antagonists into the CSF (intrathecal administration) is also a form of localized anatomic administration and can be accomplished by the perispinal route.

One of the advantages of perispinal delivery is that administration is simplified. For example, administration for the treatment of an annular tear of an intervertebral disc in the lumbar spine is effective by the interspinous route adjacent to the involved disc. This route is simple and safe. Hemorrhage due to the use of long or large bore needles is minimized because perispinal administration, by the subcutaneous route, requires only a short, narrow bore needle. Time-consuming and difficult epidural injection is not necessary. Epidural administration, for the purposes of this patent, is also a form of perispinal administration, and, in certain clinical circumstances may be the delivery method of choice, despite its greater difficulty and greater risk. Local perispinal administration also has the advantage of providing a depot of therapeutic medication in the surrounding tissue, which will provide therapeutic levels of medication to the treatment site for a prolonged period of time. This decreases the necessity for another injection of medication. Additionally, administering medication locally limits the exposure of the medication to the systemic circulation, thereby decreasing renal and hepatic elimination of the medication, and decreasing exposure of the medication to systemic metabolism. All of these factors tend to increase the therapeutic half-life of the administered cytokine antagonist. Intranasal administration is also a form of localized anatomic administration. It shares the above advantages with perispinal administration, and has the additional advantage of delivering the biologic to the area (upper nasal passages) directly adjacent to the brain. Additionally the biologics are delivered in this same manner directly to branches of the olfactory nerve, providing another route of delivery to the CNS. Taken together, all of these forms of localized anatomic administration have significant clinical advantages over the various forms of systemic administration previously used to deliver these cytokine antagonists. These forms of systemic administration include the intravenous route; the intramuscular route, when the site of intramuscular administration is remote from the site of pathology; the subcutaneous route, when the site of subcutaneous administration is remote from the site of pathology (such as an abdominal, thigh, or arm administration for the treatment of sciatica); or other methods of administration which rely on the use of the systemic circulation to deliver the medication to the target area of pathology.

For the sake of this invention, the following definitions also apply: perilesional is defined as in anatomic proximity to the site of the pathologic process being treated; and peridural is defined as in anatomic proximity to the dura of the spinal cord. The “interspinous route” for the purposes of this patent, is defined as parenteral injection through the skin in the midline, in the interspace between two spinous processes, to deliver the therapeutic molecule in anatomic proximity to the spine.

Biologics to be used for the purposes of this patent fall into the general categories of TNF antagonists or interleukin-1 antagonists.

TNF antagonists include, but are not limited to the following: etanercept (Enbrel®-Amgen); infliximab (Remicade®-Johnson and Johnson); D2E7 (Humira), a human anti-TNF monoclonal antibody (Abbott Laboratories); CDP 571 (a humanized anti-TNF IgG4 antibody); CDP 870 (an anti-TNF alpha humanized monoclonal antibody fragment), both from Celltech; soluble TNF receptor Type I (Amgen); pegylated soluble TNF receptor Type I (PEGs TNF-R1) (Amgen); and onercept, a recombinant TNF binding protein (r-TBP-1) (Serono).

In one preferred embodiment a patient with complaining of premenstrual syndrome or primary dysmenorrhea is treated by injection of a TNF antagonist selected from the group of etanercept, infliximab, CDP 870, D2E7, or onercept in a therapeutically effective dose to the anatomic area adjacent to the spine.

In one preferred embodiment a patient with premenstrual syndrome or primary dysmenorrhea is treated by injection of a TNF antagonist selected from the group of etanercept, infliximab, CDP 870, D2E7, or onercept in a therapeutically effective dose to the anatomic area adjacent to the spine, with the dose repeated as a form of chronic therapy at intervals as often as twice per week to as little as once per year.

In another preferred embodiment a patient with premenstrual syndrome or primary dysmenorrhea is treated by intranasal administration of a TNF antagonist selected from the group of etanercept, infliximab, CDP 870, D2E7, or onercept in a therapeutically effective dose.

In another preferred embodiment injection of the therapeutic molecule to the anatomic area adjacent to the spine is accomplished by interspinous injection.

In another preferred embodiment interspinous injection is accomplished by injection through the skin in the anatomic area between two adjacent spinous processes of the vertebral column.

An example of one preferred embodiment for treatment of premenstrual syndrome or primary dysmenorrhea is the perispinal administration of etanercept 25 mg by injecting through the skin of the back, between the L3 and L4 spinous processes, to deliver etanercept in anatomic proximity to the site of disc herniation.

Scientific Background:

Antibodies (immunoglobulins) are proteins produced by one class of lymphocytes (B cells) in response to specific exogenous foreign molecules (antigens). Monoclonal antibodies (mAB), identical immunoglobulin copies which recognize a single antigen, are derived from clones (identical copies) of a single B cell. This technology enables large quantities of an immunoglobulin with a specific target to be mass produced.

Monoclonal antibodies with a high affinity for a specific cytokine will tend to reduce the biologic activity of that cytokine. Substances which reduce the biologic effect of a cytokine can be described in any of the following ways: as a cytokine blocker; as a cytokine inhibitor; or as a cytokine antagonist. In this patent, the terms blocker, inhibitor, and antagonist are used interchangeably with respect to cytokines.

Advances in biotechnology have resulted in improved molecules as compared to simply using monoclonal antibodies. One such molecule is CDP 870 which, rather than being a monoclonal antibody, is a new type of molecule, that being an antibody fragment. By removing part of the antibody structure, the function of this molecule is changed so that it acts differently in the human body. Another new type of molecule, distinct from monoclonal antibodies and soluble receptors, is a fusion protein. One such example is etanercept. This molecule has a distinct function which acts differently in the human body than a simple soluble receptor or receptors.

Monoclonal antibodies, fusion proteins, and all of the specific molecules discussed above under the categories of TNF antagonists and interleukin antagonists are considered biologics, in contrast to drugs that are chemically synthesized. These biologics are derived from living sources (such as mammals (including humans), other animals, and microorganisms). The biologics mentioned above are manufactured using biotechnology, which usually involves the use of recombinant DNA technology. Cytokine antagonists are one type of biologic. Biologics are regulated through a specific division of the FDA.

Cytokine antagonists can take several forms. They may be monoclonal antibodies (defined above). They may be a monoclonal antibody fragment. They may take the form of a soluble receptor to that cytokine. Soluble receptors freely circulate in the body. When they encounter their target cytokine they bind to it, effectively inactivating the cytokine, since the cytokine is then no longer able to bind with its biologic target in the body. An even more potent antagonist consists of two soluble receptors fused together to a specific portion of an immunoglobulin molecule (Fc fragment). This produces a dimer composed of two soluble receptors which have a high affinity for the target, and a prolonged half-life. This new molecule is called a fusion protein. An example of this new type of molecule, called a fusion protein, is etanercept (Enbrel).

Tumor necrosis factor (TNF), a naturally occurring cytokine present in humans and other mammals, plays a key role in the inflammatory response, in the immune response and in the response to infection. TNF is formed by the cleavage of a precursor transmembrane protein, forming soluble molecules which aggregate in vivo to form trimolecular complexes. These complexes then bind to receptors found on a variety of cells. Binding produces an array of pro-inflammatory effects, including release of other pro-inflammatory cytokines, including IL-6, IL-8, and IL-1; release of matrix metalloproteinases; and up regulation of the expression of endothelial adhesion molecules, further amplifying the inflammatory and immune cascade by attracting leukocytes into extravascular tissues.

Etanercept (Enbrel, Amgen), infliximab (Remicade), D2E7(adalimumab), CDP 870, and onercept are potent and selective inhibitors of TNF. CDP 870 and onercept are in clinical development. Etanercept, adalimumab, and infliximab are FDA approved for chronic systemic use to treat rheumatoid arthritis.

Perispinal administration and intranasal administration of cytokine antagonists are new methods of administration of the specific cytokine antagonists of consideration here. These new methods result in improved delivery of these therapeutic molecules to the nervous system, either by local diffusion; by improved transport into the cerebrospinal fluid (CSF); or by direct transport into the CNS. Improved delivery thereby enables these specific cytokine antagonists to produce therapeutic benefit for patients with premenstrual syndrome or primary dysmenorrhea.

Clinical Disorders

Patients with the following clinical disorders, among others, will benefit from treatment with cytokine antagonists delivered by the perispinal route or by intranasal administration:

1. Unipolar and Bipolar Affective Disorders

These are disorders of mood, causing recurrent depression and/or recurrent episodes of mood elevation, resulting in mania or hypomania. Current treatment regimens include the use of lithium carbonate, carbamazepine, or anti-psychotic medication. Inflammatory cytokines are involved in the regulation of sleep and mood. In the present invention, perispinal administration of TNF antagonists or IL-1 antagonists is used for the acute or chronic treatment of these disorders. Clinical experience has demonstrated the rapid beneficial effect, and the lasting beneficial effect, of this method of treatment for these disorders. Acute administration of a TNF antagonist results in rapid improvement in affect and cognitive function. Chronic administration results in decreased lability of mood, increased time intervals between mood swings, and decreased amplitude of mood swings. Chronic administration may require twice weekly dosing, but in some patients will be effective when given much less often, sometimes as little as once per three months. Some patients may only require a single dose given at the onset of a mood disturbance. Sleep improvement and improvement in cognition is noted by patients responding to treatment.

2. Depression

Clinical depression is characterized by depressed mood, often accompanied by additional clinical manifestations, such as sleep disturbance, weight loss, loss of appetite, apathy, anhedonia, and when severe, can be associated with suicidal ideation. It is currently treated, when indicated, with antidepressant medication, most commonly selective serotonin reuptake inhibitors (SSRIs) or tricyclic antidepressants. Post-partum depression can be especially serious, occurring after childbirth. Depression, even when treated, is associated with an increased suicide risk. These patients have a disturbance in cytokine patterns, which is amenable to treatment with TNF or IL-1 antagonists by perispinal administration or by intranasal administration. Clinical experience has demonstrated the rapid beneficial effect, and the lasting beneficial effect, of this method of treatment for these disorders. Treatment can be acute or chronic, as outlined in the discussion of unipolar and bipolar affective disorder.

3. Anorexia Nervosa

Anorexia Nervosa is an eating disorder characterized by refusal to maintain body weight above a minimally normal weight (usually defined as 85% of expected), combined with a disturbance in the way one's weight or body shape is experienced and intense fear of gaining weight. This is associated with a disturbance in cytokine patterns, which is amenable to treatment with TNF or IL-1 antagonists by perispinal administration or by intranasal administration. Clinical experience has demonstrated weight gain as a result of the use of TNF antagonists.

4. Obsessive-Compulsive Disorder (OCD)

OCD is an anxiety disorder characterized by persistent intrusive thoughts that can only be alleviated by patterns of rigid and ceremonial behavior. Traditional treatment may include the use of SSRIs but is often unsuccessful. These patients have a disturbance in cytokine patterns, which is amenable to treatment with TNF or IL-1 antagonists by perispinal administration or by intranasal administration.

5. Headache Syndromes Including Migraine Headaches and Cluster Headaches

Elevated levels of inflammatory cytokines are found in patients with severe neurologic headache syndromes, including, but not limited to migraine headaches and cluster headaches. Migraine headaches, a form of vascular headache, are common, and may have associated neurologic symptoms, such as visual disturbance, photophobia, and, in rare instances, can be associated with stroke. Treatment of these disorders with TNF antagonists or IL-1 antagonists by perispinal administration leads to clinical improvement. Treatment regimens can be either acute or chronic, and will vary with the clinical setting. Clinical experience has demonstrated the beneficial effect of this method of treatment for these disorders, often with rapid diminution of headache pain demonstrated.

6. Neuropathic Pain

TNF has been found to be of central importance in the pathogenesis of several types of neuropathic pain, including, but not limited to spinal radiculopathy, nerve root inflammation due to intervertebral disc herniation, and neuropathy associated with chronic constriction injury. There are many other forms of neuropathic pain, defined generally as pain initiated or caused by a primary lesion or dysfunction in the nervous system. Treatment of these disorders with TNF antagonists or IL-1 antagonists by perispinal administration leads to clinical improvement. Treatment regimens can be either acute or chronic, and will vary with the clinical setting. Clinical experience has demonstrated the beneficial effect of this method of treatment for several different forms of neuropathic pain.

7. Lumbar and Cervical Radiculopathy

Inflammation of the nerve roots in the lumbar or cervical region may lead to neurologic dysfunction. These forms of radiculopathy commonly result in pain in a nerve root distribution, often with sensory dysfunction characterized by numbness and/or paresthesia. A smaller subset of these patients also experience motor weakness. TNF has been strongly implicated in the pathogenesis of these clinical syndromes. Release of TNF from damaged intervertebral discs, as the result of disc herniation or other forms of disc disease has been suggested to be the central causative factor. Clinical experience has established the efficacy of treatment of these disorders with TNF antagonists delivered by perispinal administration.

8. Fibromyalgia

Fibromyalgia is a syndrome of unknown cause that results in chronic, widespread neuromuscular pain and fatigue, often with multiple, tender areas, sleep disturbance, and additional clinical symptoms. Clinical experience has established the efficacy of treatment of patients with this diagnosis utilizing TNF antagonists delivered by perispinal administration. Treatment with TNF antagonists or IL-1 antagonists given parenterally by perispinal administration leads to clinical improvement.

9. Low Back Pain

Low back pain (LBP) can result from a wide variety of clinical conditions. Many forms of LBP are mild or spontaneously resolve. Other types are severe, treatment refractory, and can either be acute, subacute or chronic. Many of these patients have been diagnosed with intervertebral disc disease, ranging from a solitary annular tear of one disc capsule, to a mild disc bulge, to multiple large disc herniations present in a single individual. Clinical experience has established the efficacy of treatment of patients with these disc disorders through the use of TNF antagonists delivered by perispinal administration. In addition this method of treatment has been beneficial for other patients with back pain, including those patients with apparently normal MRI examination of the spine. Many of these patients may have undiagnosed annular tears of their intervertebral disc capsules, or other forms of internal disc derangement. Treatment with TNF antagonists or IL-1 antagonists given parenterally by perispinal administration leads to clinical improvement.

10. Chronic Fatigue Syndrome (CFS)

Patients with CFS have severe chronic fatigue of six months or longer duration, with known causes excluded; and have additional symptoms, including memory impairment, sore throat, adenopathy, myalgias, arthralgias, and sleep disturbance. Treatment with TNF antagonists or IL-1 antagonists by perispinal administration or intranasal administration leads to clinical improvement. Clinical experience has documented the favorable effect of this method of treatment for patients with this diagnosis.

11. Pre-Menstrual Syndrome

Pre-menstrual syndrome, also called late luteal phase syndrome, or premenstrual dysphoric disorder, often involves recurrent monthly episodes of changes in mood and behavior, sometimes accompanied by physiologic changes, such as weight gain. Inflammatory cytokines are involved in the pathophysiology of this disorder. Treatment with a biologic TNF inhibitor given either parenterally by perispinal administration or by normal systemic (parenteral) administration leads to clinical improvement. Biologic TNF inhibitors of consideration here include, but are not limited to, all of the following types: A monoclonal anti-TNF antibody; a fusion protein containing a TNF receptor or receptors; a monoclonal anti-TNF antibody fragment; or a TNF binding protein. Examples of these biologic TNF inhibitors include, but are not limited to: etanercept (Enbrel®, Amgen), infliximab (Remicade®, Johnson and Johnson), CDP571 (Celltech) (a humanized monoclonal anti-TNF-alpha IgG4 antibody), CDP 870 (Celltech) (a humanized monoclonal anti-TNF-alpha antibody fragment), D2E7(Humira®, Abbott Laboratories) (a human anti-TNF mAb), soluble TNF receptor Type I, pegylated soluble TNF receptor Type I (PEGs TNF-R1)(Amgen) and onercept, a recombinant TNF binding protein (r-TBP-1) (Serono). For any of these biologics the dosage utilized is in the same range as used to treat rheumatoid arthritis, but can vary from one-quarter to twice the amount for each dose i.e. if the usual dose for rheumatoid arthritis is 100 mg, then the dosage utilized here varies from 25 mg to 200 mg. The dosage schedule can vary from a single monthly dose, administered prior to menstruation, or, for more severely affected individuals, the same schedule as used to treat rheumatoid arthritis. For example, for etanercept, this means dosing as often as twice per week, 25 mg per dose, although most patients will require only a single dose per month, 25 mg, usually given about five days prior to the onset of menstruation. Some patients have benefitted from a single dose given once every two or three months.

12. Primary Dysmenorrhea

Dysmenorrhea is defined as painful menstruation. Primary dysmenorrhea is defined as painful menstruation which is unassociated with macroscopic pelvic pathology. Inflammatory cytokines are involved in the pathophysiology of this disorder. Treatment with TNF antagonists given either parenterally by perispinal administration or by normal systemic (parenteral) administration leads to clinical improvement. Biologic TNF inhibitors of consideration here include, but are not limited to, all of the following types: A monoclonal anti-TNF antibody; a fusion protein containing a TNF receptor or receptors; a monoclonal anti-TNF antibody fragment; or a TNF binding protein. Examples of these biologic TNF inhibitors include, but are not limited to: etanercept (Enbrel®, Amgen), infliximab (Remicade®, Johnson and Johnson), CDP571 (Celltech) (a humanized monoclonal anti-TNF-alpha IgG4 antibody), CDP 870 (Celltech) (a humanized monoclonal anti-TNF-alpha antibody fragment), D2E7 (Humira®, Abbott Laboratories) (a human anti-TNF mAb), soluble TNF receptor Type I, pegylated soluble TNF receptor Type I (PEGs TNF-R1)(Amgen) and onercept, a recombinant TNF binding protein (r-TBP-1) (Serono). For any of these biologics the dosage utilized is in the same range as used to treat rheumatoid arthritis, but can vary from one-quarter to twice the amount for each dose i.e. if the usual dose for rheumatoid arthritis is 100 mg, then the dosage utilized here varies from 25 mg to 200 mg. The dosage schedule can vary from a single monthly dose, administered just prior to, or during, menstruation, or, for more severely affected individuals, the same schedule as used to treat rheumatoid arthritis. For example, for etanercept, this means dosing as often as twice per week, 25 mg per dose, although most patients will require only a single dose per month, 25 mg, usually given just prior to, or at the onset of, menstruation. Some patients have benefitted from a single dose given once every two or three months.

Dosages and Routes of Administration

The dosage of a cytokine antagonist used for perispinal administration will in general be within one order of magnitude of the dosage used as a single dose for systemic administration. For example, if the usual dose when administered systemically is 100 mg, then the dose used for perispinal administration will usually be between 10 mg and 100 mg. The exception to this general guideline occurs with intrathecal injections or intranasal administration, where the required dosage is smaller, usually in the range of 1% to 10% of the corresponding systemic dose for the intrathecal route, and usually in the range of 10% to 25% for the intranasal route.

For the treatment of acute or severe conditions, the dose will generally be adjusted upward. In the above example the dose selected would therefore be 100 mg, rather than 10 mg, if the condition were acute and/or severe.

Localized perilesional injection can allow the use of subcutaneous administration even in the case when the medication is normally administered intravenously. An example of this would be the use of infliximab subcutaneously in the interspinous area for the treatment of premenstrual syndrome or primary dysmenorrhea.

For treating the above diseases with the above mentioned TNF antagonists, these TNF antagonists may be administered by the following routes:

The above TNF antagonists may be administered subcutaneously in the human and the dosage level is in the range of 1 mg to 300 mg per dose, with dosage intervals as short as one day.

The above TNF antagonists may be administered intramuscularly in the human and the dosage level is in the range of 1 mg to 200 mg per dose, with dosage intervals as short as two days.

The above TNF antagonists may be administered epidurally in the human and the dosage level is in the range of 1 mg to 300 mg per dose, with dosage intervals as short as two days.

The above TNF antagonists may be administered peridurally in the human and the dosage level is in the range of 1 mg to 300 mg per dose, with dosage intervals as short as two days.

The above TNF antagonists may be administered by interspinous injection in the human and the dosage level is in the range of 1 mg to 300 mg per dose, with dosage intervals as short as two days.

The above TNF antagonists may be administered by intranasal administration utilizing a nasal spray or nasal inhaler in the human and the dosage level is in the range of 1 mg to 50 mg per dose, with dosage intervals as short as four hours.

### ADVANTAGES OF THE PRESENT INVENTION

Accordingly, an advantage of the present invention is that it provides for the localized administration of specific biologics as a new pharmacologic treatment of premenstrual syndrome or primary dysmenorrhea; such that the use of these cytokine antagonists will result in the amelioration of these conditions.

Another advantage of the present invention is that it provides for specific biologics delivered by anatomically localized administration, which, when compared to systemic administration, produces one or more of the following: greater efficacy; more rapid onset; longer duration of action; improved delivery to the CNS; or fewer side effects.

Another advantage of the present invention is that it provides for specific biologics for providing suppression and inhibition of the action of cytokines in a human to treat premenstrual syndrome or primary dysmenorrhea.

Another advantage of the present invention is that it provides for specific biologics administered by specific methods for treating humans with premenstrual syndrome or primary dysmenorrhea which due to their biologic action will produce clinical improvement in the patient and will give the patient a better opportunity to heal, slow disease progression, improve mood, reduce pain, or otherwise improves the patient's health.

Another advantage of the present invention is that it provides for specific biologics, including cytokine antagonists to tumor necrosis factor alpha, using localized administration, including perispinal administration, as the preferred form of administration, for the treatment of premenstrual syndrome or primary dysmenorrhea.

Another advantage of the present invention is that it provides for specific biologics, including cytokine antagonists to tumor necrosis factor alpha or to interleukin-1, using localized administration, including perispinal administration or intranasal administration, as the preferred form of delivery, for the treatment of neuropsychiatric disorders, including depression, premenstrual syndrome or primary dysmenorrhea, anorexia nervosa and chronic fatigue syndrome.

A latitude of modification, change, and substitution is intended in the foregoing disclosure, and in some instances, some features of the invention will be employed without a corresponding use of other features. Accordingly, it is appropriate that the appended claims be construed broadly and in a manner consistent with the spirit and scope of the invention herein.

### GENERAL REFERENCES

[1]. Bathon J M, Martin R W, Fleischmann R M, et al. A comparison of etanercept and methotrexate in patients with early rheumatoid arthritis. N Engl J Med (2000);343:1586-1593.

[2]. Mease P J, Goffe B S, Metz J, VanderStoep A, Finck B, Burge D J. Etanercept in the treatment of psoriatic arthritis and psoriasis: a randomised trial. Lancet. (2000) Jul 29;356(9227):385-90.

[3]. Gorman J D, Sack K E, David J C. Treatment of ankylosing spondylitis by inhibition of tumor necrosis factor-alpha. N Engl J Med (2000);346:1349-1356.

[4]. Moreland L W, Schiff M H, Baumgarmer S W, et al. Etanercept therapy in rheumatoid arthritis: a randomized, controlled trial. N Engl J Med (1999);130:478-486.

[5]. Weinblatt M E, Kremer J M, Bankhurst A D, et al. A trial of etanercept, a recombinant tumor necrosis factor receptor: Fc Fusion protein, in patients with rheumatoid arthritis receiving methotrexate. N Engl J Med (1999);340(4):253-259.

[6]. Lovell D J, Giannini E H, Reiff A, et al. Etanercept in children with polyarticular juvenile rheumatoid arthritis. N Engl J Med (2000);342:763-769.

What is claimed is:

1. A method for treating premenstrual syndrome in a human by inhibiting the action of tumor necrosis factor (TNF) through the administration of a TNF inhibitor comprising the step of:
a) administering a therapeutically effective dosage level to said human of said TNF inhibitor selected from the group consisting of etanercept, infliximab, CDP571 (a humanized monoclonal anti-TNF-alpha IgG4 antibody), CDP870 (a humanized monoclonal anti-TNF-alpha antibody fragment), D2E7 (a human anti-TNF mAb), soluble TNF receptor Type I, pegylated soluble TNF receptor Type I (PEGs TNF-R1) and onercept, a recombinant TNF binding protein (r-TBP-1) (Serono).

2. A method for treating premenstrual syndrome in a human by inhibiting the action of tumor necrosis factor (TNF) through the administration of a TNF inhibitor comprising the steps of:
a) administering a therapeutically effective dosage level to said human of said TNF inhibitor selected from the group consisting of etanercept, infliximab, CDP571 (a humanized monoclonal anti-TNF-alpha IgG4 antibody), CDP870 (a humanized monoclonal anti-TNF-alpha antibody fragment), D2E7 (a human anti-TNF mAb), soluble TNF receptor Type I, pegylated soluble TNF receptor Type I (PEGs TNF-R1) and onercept, a recombinant TNF binding protein (r-TBP-1) (Serono); and
b) administering said dose parenterally by perispinal administration.

3. A method for treating premenstrual syndrome in a human by inhibiting the action of tumor necrosis factor (TNF) through the administration of a biologic TNF inhibitor comprising the step of:
a) administering a therapeutically effective dosage level to said human of said biologic TNF inhibitor selected from the group consisting of: a monoclonal anti-TNF antibody; a fusion protein containing a TNF receptor or receptors; a monoclonal anti-TNF antibody fragment; or a TNF binding protein.

4. A method for treating primary dysmenorrhea in a human by inhibiting the action of tumor necrosis factor (TNF) through the administration of a TNF inhibitor comprising the step of:
a) administering a therapeutically effective dosage level to said human of said TNF inhibitor selected from the group consisting of etanercept, infliximab, CDP571 (a humanized monoclonal anti-TNF-alpha IgG4 antibody), CDP870 (a humanized monoclonal anti-TNF-alpha antibody fragment), D2E7 (a human anti-TNF mAb), soluble TNF receptor Type I, pegylated soluble TNF receptor Type I (PEGs TNF-R1) and onercept, a recombinant TNF binding protein (r-TBP-1) (Serono).

5. A method for treating primary dysmenorrhea in a human by inhibiting the action of tumor necrosis factor (TNF) through the administration of a TNF inhibitor comprising the steps of:
a) administering a therapeutically effective dosage level to said human of said TNF inhibitor selected from the group consisting of etanercept, infliximab, CDP571 (a humanized monoclonal anti-TNF-alpha IgG4 antibody), CDP870 (a humanized monoclonal anti-TNF-alpha antibody fragment), D2E7 (a human anti-TNF mAb), soluble TNF receptor Type I, pegylated soluble TNF receptor Type I (PEGs TNF-R1) and onercept, a recombinant TNF binding protein (r-TBP-1) (Serono); and
b) administering said dose parenterally by perispinal administration.

6. A method for treating primary dysmenorrhea in a human by inhibiting the action of tumor necrosis factor (TNF) through the administration of a biologic TNF inhibitor comprising the step of:
a) administering a therapeutically effective dosage level to said human of said biologic TNF inhibitor selected from the group consisting of: a monoclonal anti-TNF antibody; a fusion protein containing a TNF receptor or receptors; a monoclonal anti-TNF antibody fragment; or a TNF binding protein.
